(19) United States
(12) Patent Application Publication
(10) Pub. No.: US 2016/0310155 A1
(43) Pub. Date: Oct. 27, 2016

(54) ATRIAL-APPENDAGE LIGATION SURGICAL TOOL AND ATRIAL APPENDAGE LIGATION SYSTEM

(71) Applicant: OLYMPUS CORPORATION, Tokyo (JP)

(72) Inventors: Takehiro KIMURA, Tokyo (JP); Kazutoshi KUMAGAI, Tokyo (JP); Yoshiro OKAZAKI, Tokyo (JP); Naoya SUGIMOTO, Tokyo (JP); Hiroki HIBINO, Tokyo (JP); Yoshiyuki KUMADA, Tokyo (JP); Hiroshi FUKUDA, Tokyo (JP)

(73) Assignee: OLYMPUS CORPORATION, Tokyo (JP)

(21) Appl. No.: 15/204,231

(22) Filed: Jul. 7, 2016

Related U.S. Application Data
(63) Continuation of application No. PCT/JP2015/053850, filed on Feb. 12, 2015.

(30) Foreign Application Priority Data
Feb. 28, 2014 (JP) 2014-039815

(51) Int. Cl. A61B 17/29; A61B 34/00; A61B 17/12

(52) U.S. Cl. CPC A61B 17/29; A61B 17/12013; A61B 34/73; A61B 2017/00243

(57) ABSTRACT

An atrial-appendage ligation surgical tool that includes a pressing part and a shaft, in which the pressing part is guided into a pericardium via a sheath penetrating through a pericardial membrane, and includes contact portions that press the vicinity of a base of an atrial appendage and have a circular cross-sectional shape and round distal ends, and in which the shaft is in an inserted state in the sheath, supports the pressing part at a distal end, and has a rigidity that enables transmission of a pressing force acting in the longitudinal direction, applied from the proximal end side of the sheath.

### CROSS-REFERENCE TO RELATED APPLICATIONS

This is a continuation of International Application PCT/JP2015/053850, with an international filing date of Feb. 12, 2015, which is hereby incorporated by reference herein in its entirety. This application claims the benefit of Japanese Patent Application No. 2014-039815, filed on Feb. 28, 2014, the content of which is incorporated herein by reference.

### TECHNICAL FIELD

The present invention relates to an atrial-appendage ligation surgical tool and to an atrial appendage ligation system.

### BACKGROUND ART

The population of patients with atrial fibrillation, which is one form of cardiac arrhythmia has shown an increasing tendency in recent years. Strokes triggered by atrial fibrillation are thought to occur because a cerebral vessel becomes blocked with a thrombus that originated in the heart (mostly in a left atrial appendage). The most frequent embolic source of cardioembolic stroke is the left atrial appendage thrombus.

A therapy commonly recommended for prevention of cardioembolic stroke is anticoagulation treatment with warfarin; however, medication management of warfarin is difficult, and there is a risk of hemorrhagic complications. As a substitute therefor, a method for preventing embolisms by closure of the left atrial appendage has been developed (for example, Watchman produced by Boston Scientific). This method uses a jellyfish-shaped device that closes the left atrial appendage transvascular manner.

There is a known surgical tool that ligates an atrial appendage from outside the heart without the use of an anticoagulant or the use of transvascular procedures (for example, refer to Patent Literature 1). This is a surgical tool with which forceps and a ligation loop are inserted into a pericardium from outside the body, the ligation loop is put around the atrial appendage while the tip of the atrial appendage is being retained and pulled by the retention forceps, and then the ligation loop is tightened to ligate the atrial appendage.

This surgical tool is equipped with a sleeve that accommodates the ligation loop in a recessed portion of the sleeve. The sleeve keeps the ligation loop wide open so that the endoloop can be easily put around the atrial appendage.

### CITATION LIST

### Patent Literature

### {PTL 1}

US Patent Application Publication No. 2008/0294175 specification

### SUMMARY OF INVENTION

### Technical Problem

When atrial appendage ligation operation is performed with this surgical tool, just pulling the tip of an atrial appendage by retention forceps causes the outer wall of the left atrium to deform and move in the pulling direction together with the atrial appendage; accordingly, there is a possibility that the base of the atrial appendage, where it is desired to perform ligation, cannot be exposed.

The present invention is an atrial-appendage ligation surgical tool and an atrial appendage ligation system that facilitate ligation in the vicinity of the base of an atrial appendage with a ligation loop.

### Solution to Problem

One aspect of the present invention provides at least one atrial-appendage ligation surgical tool. The surgical tool includes a pressing part that is guided into a pericardium via a sheath penetrating through a pericardial membrane, and that includes a contact portion that presses a vicinity of a base of an atrial appendage; and a shaft that is disposed in an inserted state in the sheath and supports the pressing part at a distal end.

According to this aspect, at least one atrial-appendage ligation surgical tool is guided into the pericardium via a sheath penetrating through the pericardial membrane, and the pressing part in the pericardium can be advanced toward the atrial appendage by pushing the shaft in the longitudinal direction at the proximal side of the sheath. As a result, the distal end is placed in the vicinity of the atrial appendage, for example, under the atrial appendage. At this stage, an observation means such as an endoscope may be separately inserted and placed by using an X-ray fluoroscopic image.

Another aspect of the present invention provides an atrial-appendage ligation surgical tool that includes a pressing part that is guided into a pericardium via a sheath penetrating through a pericardial membrane, and that includes two bar-shaped contact portions that can be arranged along a width direction of the atrial appendage and at a position where the atrial appendage is pinched in a thickness direction; a shaft that is disposed in an inserted state in the sheath and that supports the pressing part at a distal end; and a biasing means for biasing the pressing part so that the pressing part is arranged in lined manner with respect to the shaft when the pressing part is inside the sheath and the pressing part extends in a direction intersecting a longitudinal axis of the shaft when the pressing part is released from the sheath.

According to this aspect, as the atrial-appendage ligation surgical tool is guided into the pericardium via the sheath penetrating through the pericardial membrane, the pressing part arranged in lined manner with respect to the shaft within the sheath is biased so that the pressing part is made to extend in a direction intersecting the longitudinal axis of the shaft by the biasing means as soon as the pressing part is released from the sheath and is in the pericardium. In this state, the pressing part is made to advance toward the atrial appendage in the pericardium by pressing the shaft in the longitudinal direction from the proximal end side of the sheath.

In the above-described aspect, the contact portions may be configured so that a spacing therebetween is changeable.

In the above-described aspect, the contact portions may be rollers that rotatable about their longitudinal axes.

In the above-described aspect, the shaft may be made of an elastic material and has a pre-bent section to constitute the biasing means.

In the aspect described above, one of the contact portions may include a magnetic material and the other contact portion may include a magnet.

In the aspect described above, the two contact portions may have retention portions that releasably retain a ligation loop guided along the shaft.

In the aspect described above, a retention portion that releasably retains a ligation loop may be provided so as to be movable along the shaft.

Another aspect of the present invention provides an atrial appendage ligation system that includes retention forceps that retain an atrial appendage and the atrial-appendage ligation surgical tool described above.

### BRIEF DESCRIPTION OF DRAWINGS

FIG. 1 is a perspective view of an atrial-appendage ligation surgical tool according to an embodiment of the present invention.

FIG. 2 is a longitudinal sectional view showing a state in which the atrial-appendage ligation surgical tool shown in FIG. 1 is housed inside a sheath.

FIG. 3 is a diagram showing a state in which a pressing part of the atrial-appendage ligation surgical tool shown in FIG. 1 is made to approach an atrial appendage in a pericardium.

FIG. 4 is a diagram showing a state in which the atrial appendage is pinched between two contact portions of the pressing part of the atrial-appendage ligation surgical tool shown in FIG. 1.

FIG. 5 is a side view showing a state in which the atrial appendage is pinched between the two contact portions of the atrial-appendage ligation surgical tool shown in FIG. 1.

FIG. 6 is a diagram illustrating how the ligation loop is placed around the atrial appendage from the state shown in FIG. 4.

FIG. 7 is a side view showing a state in which the ligation loop put around the atrial appendage in FIG. 6 is placed near the pressing part of the atrial-appendage ligation surgical tool.

FIG. 8A is a perspective view showing a rectangular-frame-shaped pressing part, which is a first modification of the pressing part of the atrial-appendage ligation surgical tool shown in FIG. 1.

FIG. 8B is a perspective view showing an oval-frame-shaped pressing part, which is a first modification of the pressing part of the atrial-appendage ligation surgical tool shown in FIG. 1.

FIG. 8C is a perspective view showing a C-shaped pressing part, which is a first modification of the pressing part of the atrial-appendage ligation surgical tool shown in FIG. 1.

FIG. 8D is a perspective view showing a V-shaped pressing part, which is a first modification of the pressing part of the atrial-appendage ligation surgical tool shown in FIG. 1.

FIG. 8E is a perspective view showing a pressing part having a shape in which only distal ends are spread out, which is a first modification of the pressing part of the atrial-appendage ligation surgical tool shown in FIG. 1.

FIG. 9 is a perspective view of a second modification of the atrial-appendage ligation surgical tool shown in FIG. 1, in which two contact portions are configured to open and close.

FIG. 10 is a front view of a third modification of the atrial-appendage ligation surgical tool shown in FIG. 1, in which the contact portions are attracted to each other by a magnet.

FIG. 11A is a perspective view showing a fourth modification of the atrial-appendage ligation surgical tool shown in FIG. 1 used to perform another method for making the shaft bend, and shows a state in which a hollow shaft is straightened by a highly rigid rod inserted therein.

FIG. 11B is a perspective view showing the fourth modification of the atrial-appendage ligation surgical tool used to perform another method for making the shaft bend, and shows a state in which the rod is pulled out so that the pre-bent section causes the shaft to bend.

FIG. 12 is a perspective view showing a fifth modification of the atrial-appendage ligation surgical tool shown in FIG. 1, in which the pressing part is bent by using a joint.

FIG. 13 is a perspective view showing a sixth modification of the atrial-appendage ligation surgical tool shown in FIG. 1, in which the direction of the bend is different from that shown in FIG. 12.

FIG. 14 is a perspective view showing a seventh modification of the atrial-appendage ligation surgical tool shown in FIG. 1, in which bending of the pressing part relative to the shaft and opening and closing of the two contact portions are both performed by using joints.

FIG. 15A is a perspective view showing an eighth modification of the atrial-appendage ligation surgical tool shown in FIG. 1, in which bending of the pressing part relative to the shaft and the opening and closing of the two contact portions are both performed by using joints, showing a state in which the pressing part extends in a straight line.

FIG. 15B is a perspective view showing the eighth modification of the atrial-appendage ligation surgical tool shown in FIG. 1, in which bending of the pressing part relative to the shaft and the opening and closing of the two contact portions are both performed by using joints, showing a state in which only contact portions are bent.

FIG. 15C is a perspective view showing the eighth modification of the atrial-appendage ligation surgical tool shown in FIG. 1, in which bending of the pressing part relative to the shaft and the opening and closing of the two contact portions are both performed by using joints, showing a state in which contact portions are opened and closed.

FIG. 16 is a ninth modification of the atrial-appendage ligation surgical tool shown in FIG. 1, in which the contact portions are rollers.

FIG. 17A is a perspective view showing a tenth modification of the atrial-appendage ligation surgical tool shown in FIG. 1, in which the overall structure of a tool that integrally retains a ligation loop is shown.

FIG. 17B is a perspective enlarged view of a retention portion of the tool that integrally retains a ligation loop according to the tenth modification of the atrial-appendage ligation surgical tool shown in FIG. 1.

FIG. 18 is a perspective view showing an eleventh modification of the atrial-appendage ligation surgical tool shown in FIG. 1, in which a guide rail for guiding a ligation loop is provided in a shaft.

FIG. 19 is a front view showing a twelfth modification of the atrial-appendage ligation surgical tool shown in FIG. 1, in which retention forceps can be moved relative to each other.

FIG. 20A is a perspective view showing a state in which the contact portions of the twelfth modification of the atrial-appendage ligation surgical tool shown in FIG. 1 are opened.

FIG. 20B is a perspective view showing a state in which the contact portions of the twelfth modification of the atrial-appendage ligation surgical tool shown in FIG. 1 are closed.

FIG. 20C is a front view showing a state in which an atrial appendage is pinched between the contact portions of the twelfth modification of the atrial-appendage ligation surgical tool shown in FIG. 1.

FIG. 20D is a front view showing a state in which an atrial appendage pinched between the contact portions of the twelfth modification of the atrial-appendage ligation surgical tool shown in FIG. 1 is folded.

FIG. 21 is a diagram showing a thirteenth modification of the atrial-appendage ligation surgical tool shown in FIG. 1.

FIG. 22 is a diagram showing a fourteenth embodiment of the atrial-appendage ligation surgical tool shown in FIG. 1.

FIG. 23 is a diagram showing a fifteenth embodiment of the atrial-appendage ligation surgical tool shown in FIG. 1.

### DESCRIPTION OF EMBODIMENT

A surgical tool 1 for atrial appendage ligation according to one embodiment of the present invention will now be described with reference to the drawings.

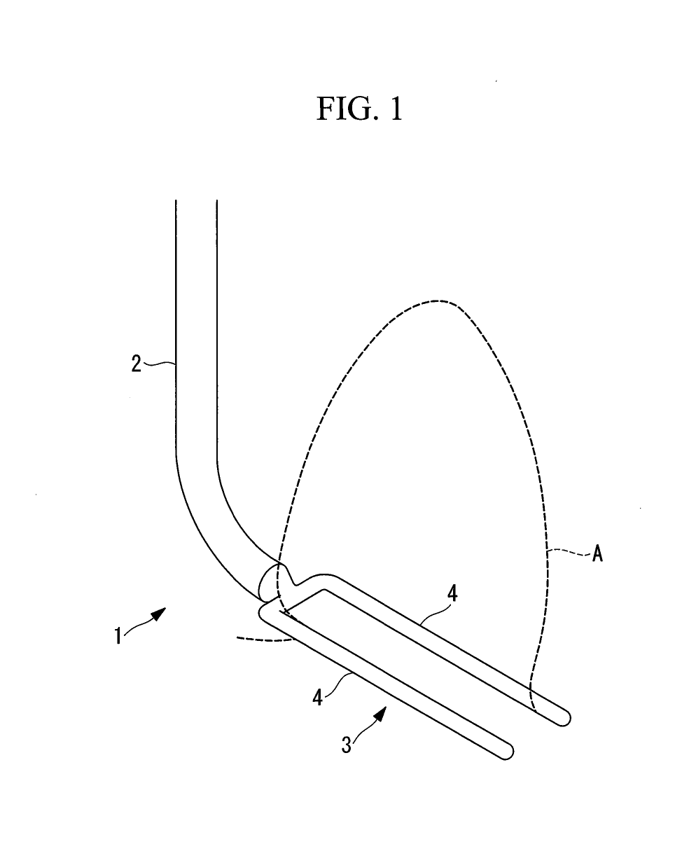

As shown in FIG. 1, the surgical tool 1 for atrial appendage ligation according to this embodiment includes an elongated shaft 2 made of an elastic material having a pre-bent section that allows a distal end of the long shaft 2 to bend 90°, and a pressing part 3 disposed at the distal end of the shaft 2. In this embodiment, the shaft 2 itself constitutes a s biasing means that biases the pressing part 3 in the direction of bending due to this pre-bent section.

The pressing part 3 includes two straight-rod-shaped contact portions 4 arranged to be parallel to each other with a particular interval therebetween, and has a bifurcated shape. The plane of a curve formed by the pre-bent section of the shaft 2 is substantially orthogonal to the plane in which the two contact portions 4 lie.

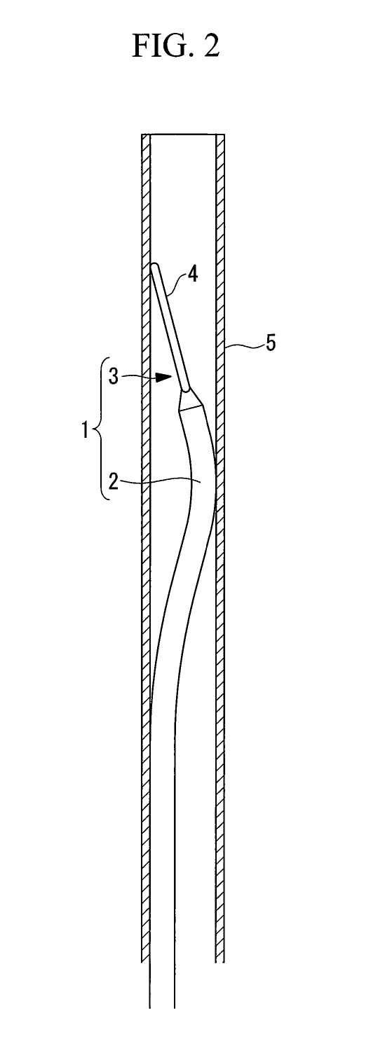

The shaft 2 can bend to follow the shape of a sheath 5, but has rigidity that enables transmission of a pressing force acting in the longitudinal direction, applied from the proximal end of the sheath 5. As shown in FIG. 2, a portion that is caused to bend due to the pre-bent section is straightened as this portion enters the sheath 5 so that the pressing part 3 can be arranged substantially in lined manner with the extension line of the shaft 2 in the longitudinal direction.

As shown in FIG. 1, each of the contact portions 4 is formed to have a circular cross-section, and the distal end of each contact portion 4 is rounded so that the distal end does not stick into the tissue against which it is pressed.

The spacing between the contact portions 4 is set to a value such that an atrial appendage A can be pinched by these two portions in the thickness direction.

The procedure for ligating the atrial appendage A by using the surgical tool 1 for atrial appendage ligation according to this embodiment, having the above-described configuration, is described below.

In order to ligate the atrial appendage A by using the surgical tool 1 for atrial appendage ligation according to this embodiment, first, a distal end opening of the sheath 5 is moved to the interior of the pericardium from the lower portion of the xiphoid process and by penetrating through the body surface tissue and the pericardial membrane. While this state is maintained, the surgical tool 1 for atrial appendage ligation having the pre-bent section of the shaft 2 substantially straightened is inserted into the sheath 5 and is made to advance toward the pericardium.

At this stage, the pressing part 3 is substantially in line with the shaft 2 and thus can advance smoothly inside the sheath 5. Once the pressing part 3 is delivered to the interior of the pericardium from the distal end opening of the sheath 5, the pre-bent section of the shaft 2, which has been restricted, is released and a bend is formed, as shown in FIG. 1. As a result, the pressing part 3 is directed in a direction intersecting the longitudinal direction of the shaft 2.

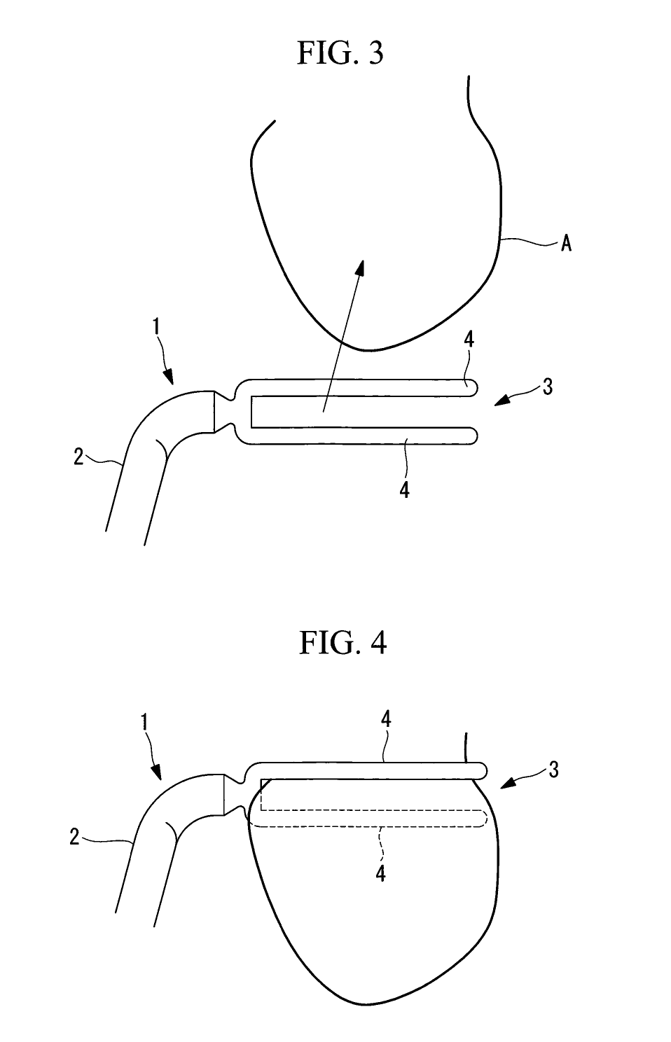

Under such conditions, the shaft 3 is operated from the proximal end of the sheath 5, which is outside the body, while conducting observation with an endoscope, which has been separately inserted into the pericardium, so that, as shown in FIG. 3, the pressing part 3 approaches the atrial appendage A from the tip side thereof, and, as shown in FIG. 4, the atrial appendage A is inserted between the two contact portions 4 of the pressing part 3. In other words, the shaft 2 is arranged to come on the right-hand side (left-hand side when viewed from the front) of the left atrial appendage. If endoscopic observation is difficult, the shaft 2 may be moved to a position other than the right-hand side of the left atrial appendage; for example, it may be moved upward. Since the atrial appendage A is bag-shaped tissue that protrudes from the outer surface of a heart like an ear, and is elastic, the atrial appendage A can be inserted between the contact portions 4 while the atrial appendage A is being pressed by the rigid pressing part 3 and deformed. Then the pressing part 3 is advanced while pulling the tip of the atrial appendage A with retention forceps (not shown) delivered via the sheath 5 so that the contact portions of the pressing part 3 can be pressed against the outer wall of the left atrium, and so that the atrial appendage A can be stretched.

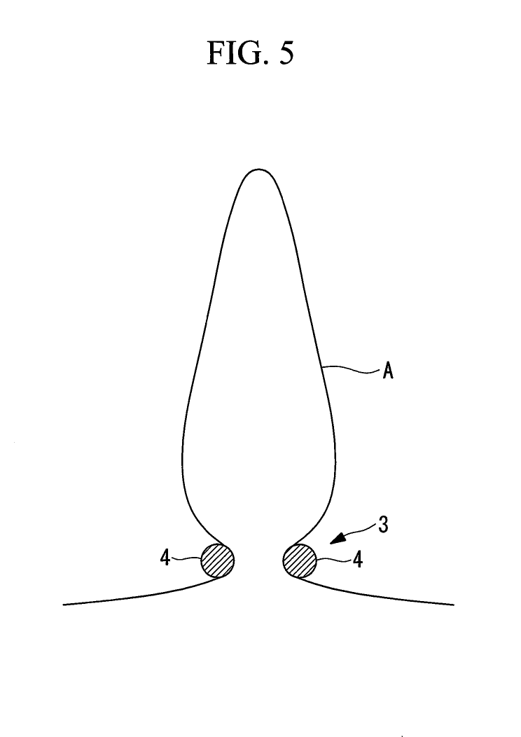

As shown in FIG. 5, the contact portions 4 are positioned near the base of the atrial appendage A, and the atrial appendage A is pinched between the contact portions 4. Thus, despite heart pulsation, the contact portions 4 pinching the atrial appendage A can stably remain pressed against the outer wall of the left atrium and can press the surface of the atrial appendage A so that the atrial appendage A is indented and does not expand in the thickness direction.

While this state is maintained, a shaft (hereinafter referred to as a loop shaft) 7 of a ligation loop 6, which has been made to approach the atrial appendage from the outer side of the retention forceps (not shown), and the retention forceps are operated simultaneously so as to put the ligation loop 6 around the atrial appendage A.

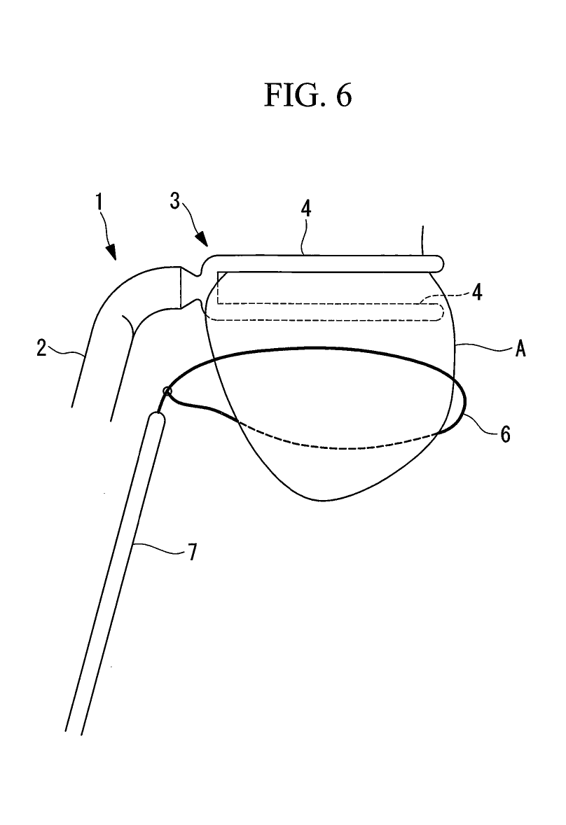

In other words, while retaining the tip of the atrial appendage A with the retention forceps, the retention forceps are pulled, and the loop shaft 7 is pushed out simultaneously so that the retention forceps retaining the atrial appendage A are removed from the inside of the ligation loop 6. As a result, as shown in FIG. 6, the atrial appendage A is inserted into the ligation loop 6, and the ligation loop 6 can be easily put around the atrial appendage A.

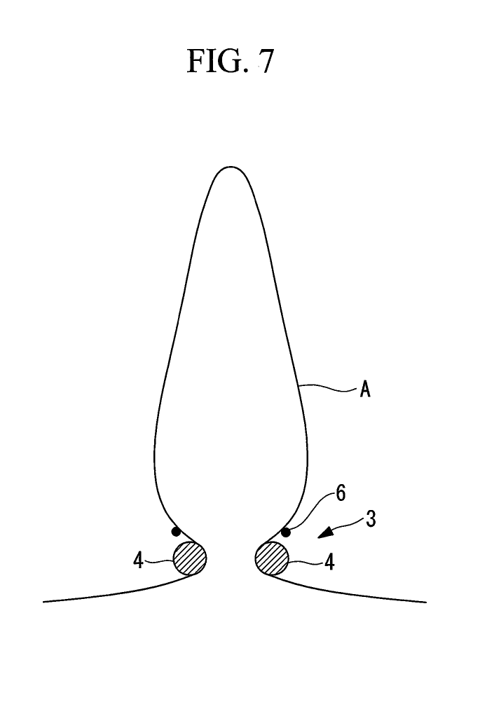

As shown in FIG. 7, as soon as the ligation loop 6 is placed at the base of the atrial appendage A exposed by being stretched and pulled by the retention forceps while the outer wall of the left atrium is being pressed by the pressing part 3, the loop shaft 7 is pushed so that one end of the ligation loop is pulled out from the proximal end side of the loop shaft 7. As a result, the ligation loop 6 is tightened and ligates the atrial appendage A.

In such a case, since the pressing part 3 of the surgical tool 1 for atrial appendage ligation is located near the position where ligation of the atrial appendage A is desirably performed, and causes the atrial appendage A to be indented in the thickness direction, tightening of the ligation loop 6 does not cause the ligation loop 6 to come off and move toward the tip of the atrial appendage A. Thus, the ligation site can be maintained.

That is, when the tip of the atrial appendage A is pulled by the retention forceps without using the surgical tool 1 for atrial appendage ligation, the outer wall of the left atrium is pulled together with the atrial appendage A. Accordingly, the atrial appendage A does not stretch enough, and the base of the atrial appendage A where ligation is desirably performed remains unexposed. In contrast, according to this embodiment, as shown in FIG. 7, the pressing part 3 pinches the portion near the base of the atrial appendage A in the thickness direction and pushes the outer wall of the left atrium; thus, the atrial appendage can be sufficiently stretched, and the base can be exposed by pulling the atrial appendage with the retention forceps. Moreover, since the surface of the atrial appendage A is indented by the pressing part 3, the ligation loop 6 is prevented from moving toward the tip and remains at the site. This is advantageous since the atrial appendage A can be ligated at a position as close to the base as possible.

After the atrial appendage A is ligated, the loop shaft 7 is pulled out while leaving the ligation loop 6 at the site, and cutting forceps, which have been guided via the sheath 5, cut the ligation loop 6 at a portion near the knot.

Subsequently, the shaft 2 of the surgical tool 1 for atrial appendage ligation is moved in a direction that enables the shaft 2 to be pulled out from the sheath, and the pressing part 3 is removed from the atrial appendage A. As the pressing part 3 is withdrawn into the sheath 5, the pre-bent section is straightened, and the pressing part 3 is removed from the body via the sheath 5.

The state of the ligated atrial appendage A is confirmed with an endoscope by moving the atrial appendage A retained by the retention forceps. If satisfactory ligation is confirmed, all surgical tools are pulled out, thus ending the procedure.

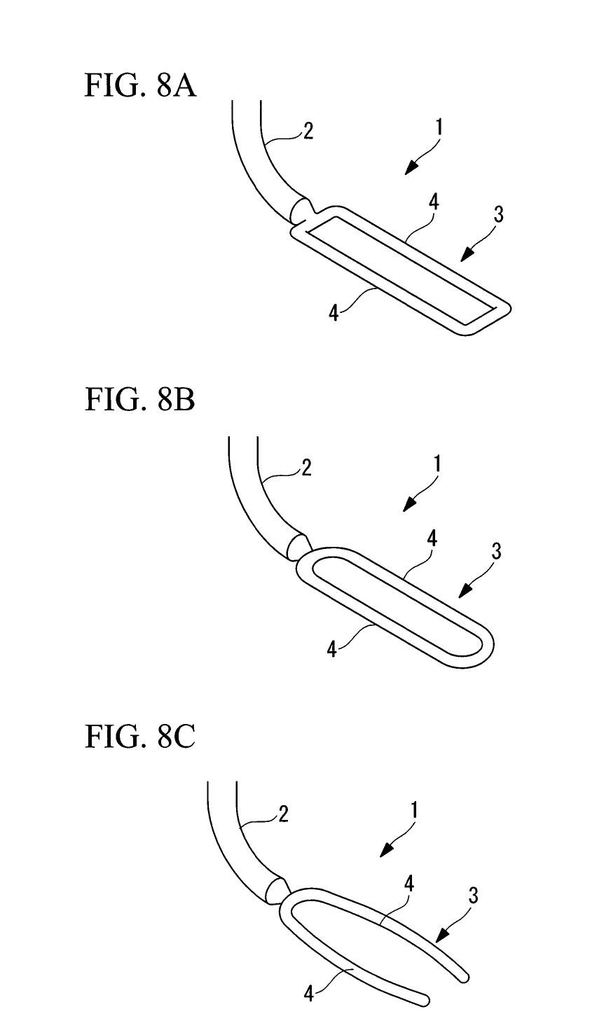

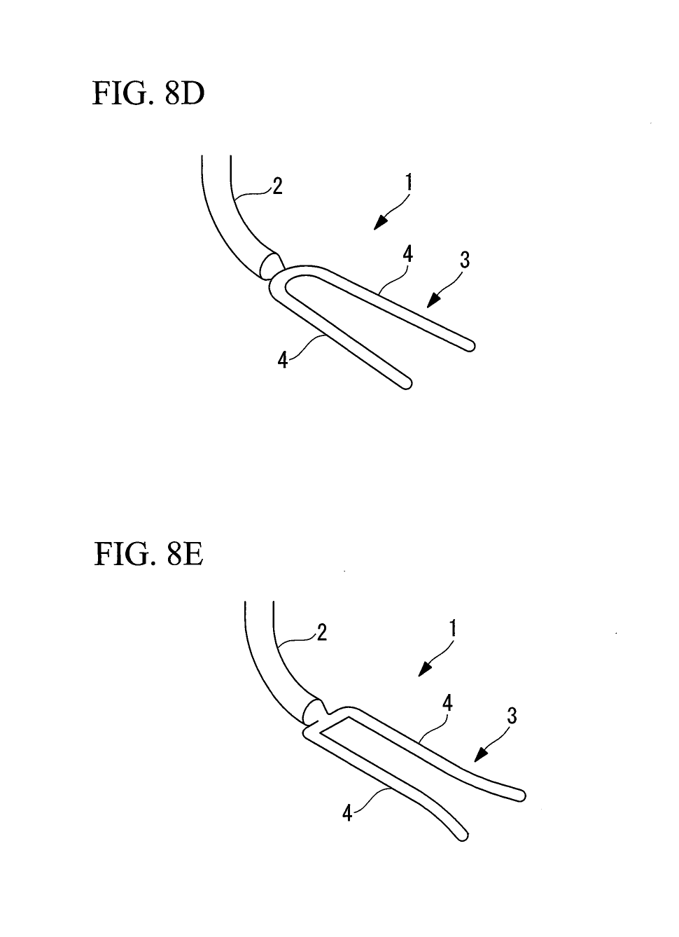

In this embodiment, the pressing part 3 having two straight-rod-shaped contact portions parallel to each other with a particular gap therebetween is described as an example. However, the invention is not limited to this structure, and any of the following forms shown in FIGS. 8A to 8E may be employed: a closed rectangular frame form (FIG. 8A) or an oval form (FIG. 8B) constituted by two straight-rod-shaped contact portions 4 having distal ends connected to each other; a substantially C-shaped form (FIG. 8C) constituted by two curved-rod-shaped contact portions 4; a substantially V-shaped form (FIG. 8D) constituted by two straight-rod contact portions 4 whose spacing increases toward the distal end; and a form (FIG. 8E) constituted by two straight-rod-shaped contact portions 4 having distal ends that spreading outward.

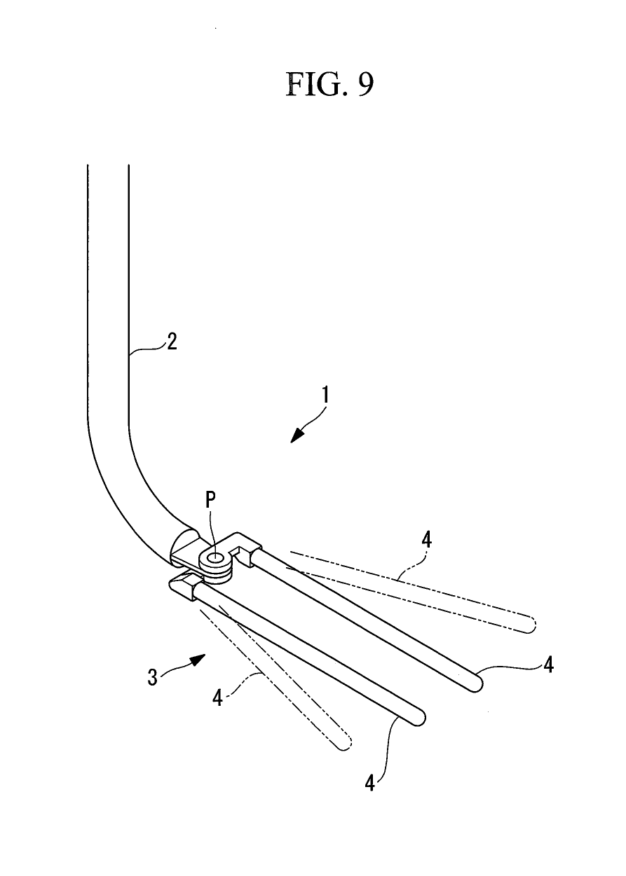

Although the pressing part 3 in which two contact portions 4 are fixed at positions that are separated from each other by a particular spacing has been described, it is possible to use an alternative, such as the one shown in FIG. 9, in which two contact portions 4 are pivoted about the axis of a joint P so that the spacing between the contact portions 4 can be changed (open or close).

According to this structure, the contact portions 4 can be brought close to each other to save space as the contact portions 4 pass via the sheath 5. In inserting the atrial appendage A, the spacing between the two contact portions can be widened to facilitate insertion. After the insertion, the two contact portions 4 can be again brought close to each other so as to pinch the atrial appendage A in the thickness direction so that the outer wall of the left atrium can be pressed while constricting the atrial appendage A.

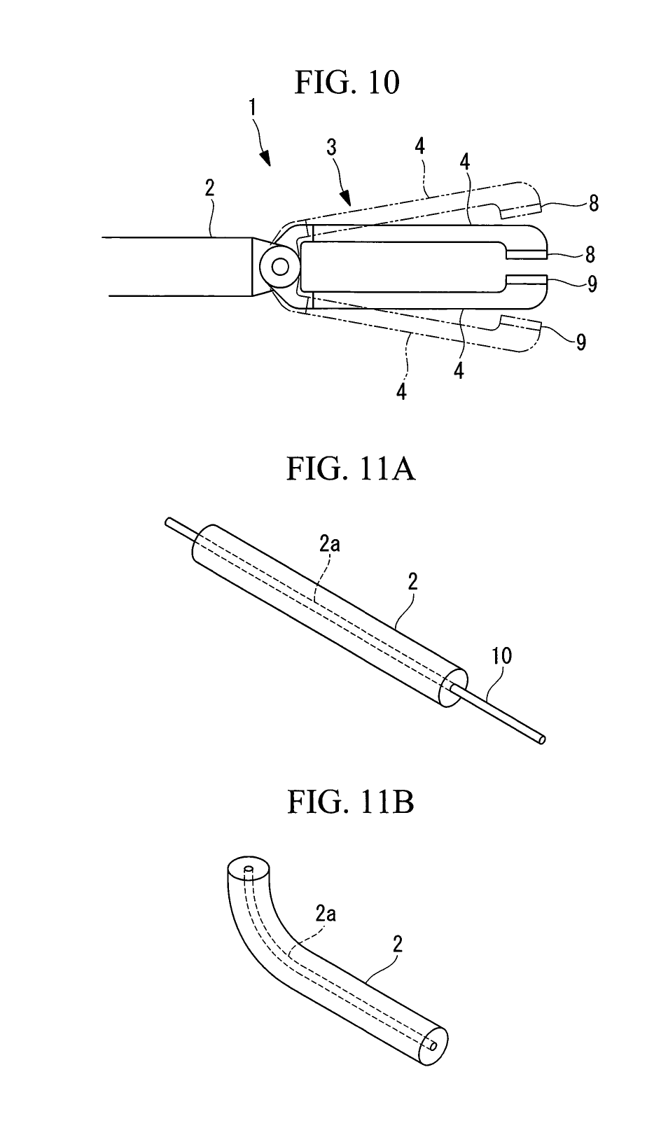

The mechanism for opening and closing the two contact portions 4 may be a wire (not shown) that has been guided along the shaft 2 and that is configured to open and close the contact portions 4, as with typical retention forceps. Alternatively, as shown in FIG. 10, a distal end 8 of one of the two contact portions 4 may be made of a magnetic material, and the other may be made of an electromagnet 9 so as to be magnetizable. The atrial appendage A is inserted while the electromagnet 6 is in an unmagnetized state, and then the electromagnet 9 is magnetized to magnetically attract the two contact portions 4 to each other so as to reliably pinch the atrial appendage A in the thickness direction and form a constriction.

Two contact portions 4 may both be equipped with electromagnets 9. When two contact portions 4 are respectively guided via different shafts 2, permanent magnets may be employed instead of the electromagnets 9.

In this embodiment, the pre-bent section of the shaft 2 is straightened by the sheath 5. Alternatively, as shown in FIG. 11A, the shaft 2 may be straightened by removably inserting a straight rod 10 having a higher rigidity than the shaft 2 into a penetrating hole 2a penetrating via the shaft 2 in the longitudinal direction of the shaft 2; and the shaft 2 may be bent by the pre-bent section by pulling out the rod 10 from the penetrating hole 2a. In this manner, compared to the case of using the sheath 5 to straighten the pre-bent section, frictional force with respect to the sheath 5 is decreased, and the ease of delivering and pulling out the shaft 2 into and from the pericardium can be improved.

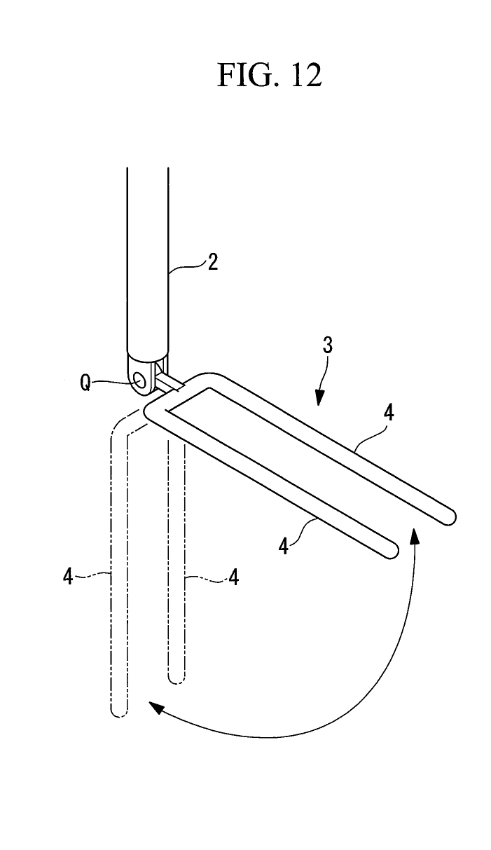

This embodiment is configured so that, by using the shaft 2, which is made of an elastic material and has a pre-bent section, the pressing part 3 in the pericardium is arranged to lie in a direction intersecting the longitudinal direction of the shaft 2. Alternatively, as shown in FIG. 12, a joint Q may be installed between the shaft 2 and the pressing part 3 so that a biasing means (not shown in the drawing), such as a spring, biases the joint Q to pivot. The pivoting direction of the joint Q is along a plane orthogonal to a plane that contains the two contact portions 4.

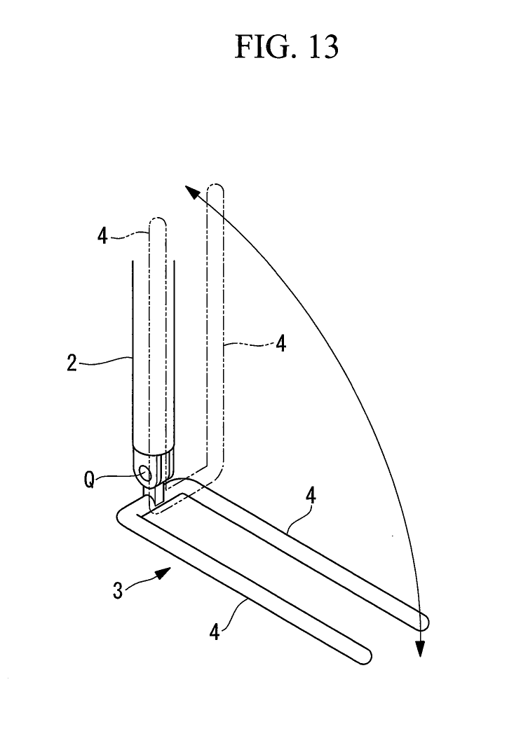

As shown by chain lines in FIG. 12, the joint Q may be made pivotable between a configuration in which the pressing part is in line with an extension line of the shaft 2 and a configuration in which the pressing part 3 is arranged at a position substantially orthogonal to the longitudinal direction of the shaft 2. Alternatively, as shown in FIG. 13, the joint Q may be made pivotable between a configuration in which the pressing part 3 is folded toward the shaft 2 and a configuration in which the pressing part 3 is arranged at a position substantially orthogonal to the longitudinal direction of the shaft 2. In the case shown in FIG. 13, a wire (not shown) for causing the pressing part 3 to return to the folded configuration is preferably provided.

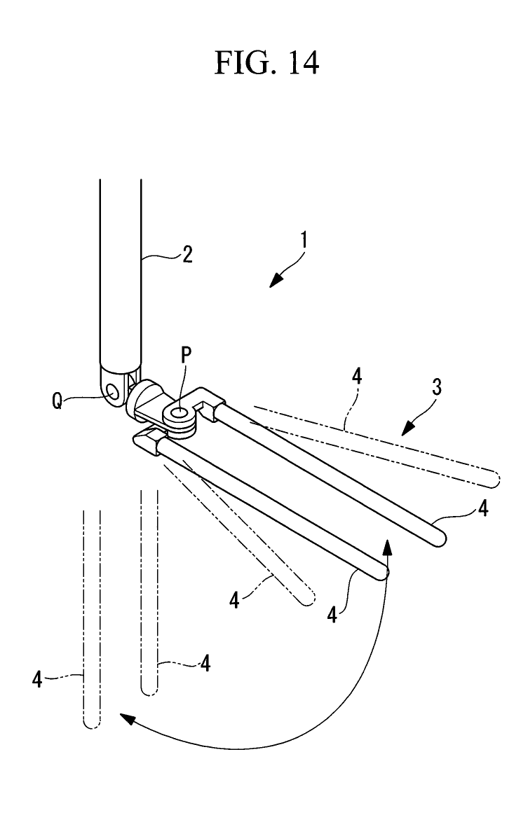

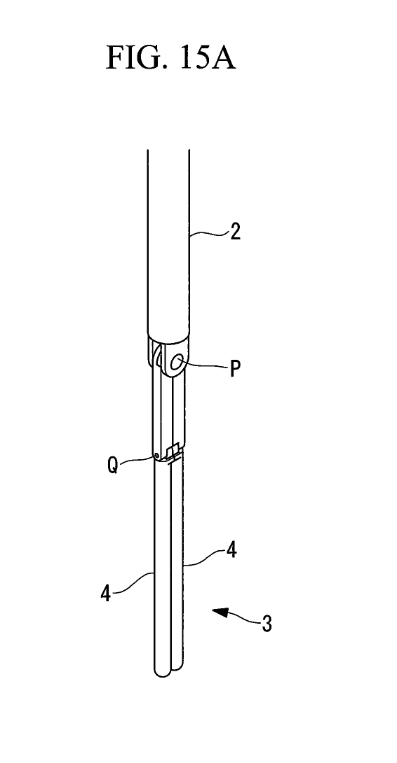

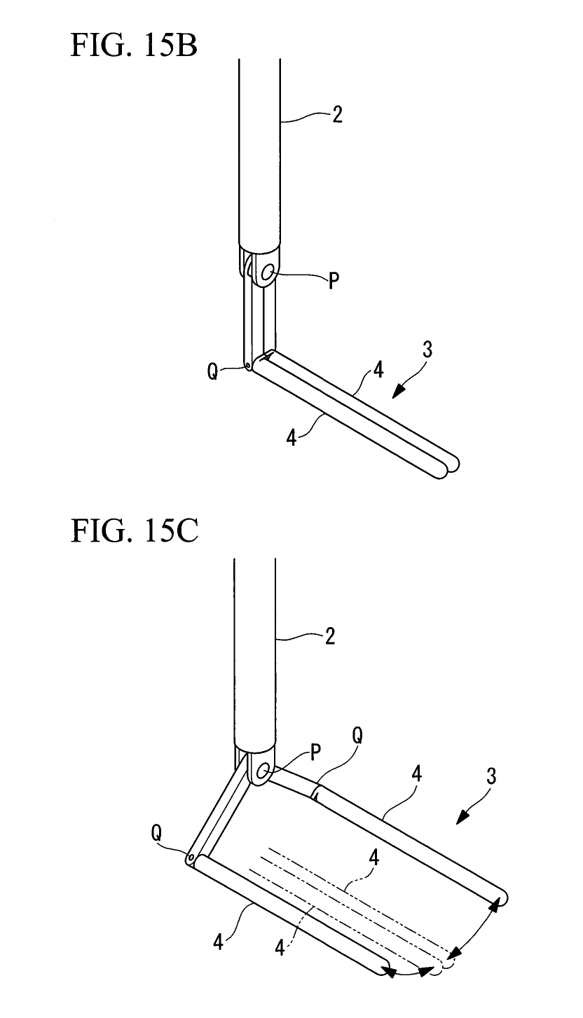

As shown in FIG. 14, in order to change the angle between the shaft 2 and the pressing part 3 by using joints P and Q and to make the pressing part 3 openable, the joint Q that causes the entire pressing part 3 to pivot relative to the shaft 2 may be installed on the proximal end side of the joint P for opening and closing the two contact portions 4 of the pressing part 3. Alternatively, as shown in FIGS. 15A to 15C, the joint P, which opens and closes the two contact portions 4, may be installed on the proximal end side of the joint Q, which causes the contact portions 4 to pivot relative to the shaft 2.

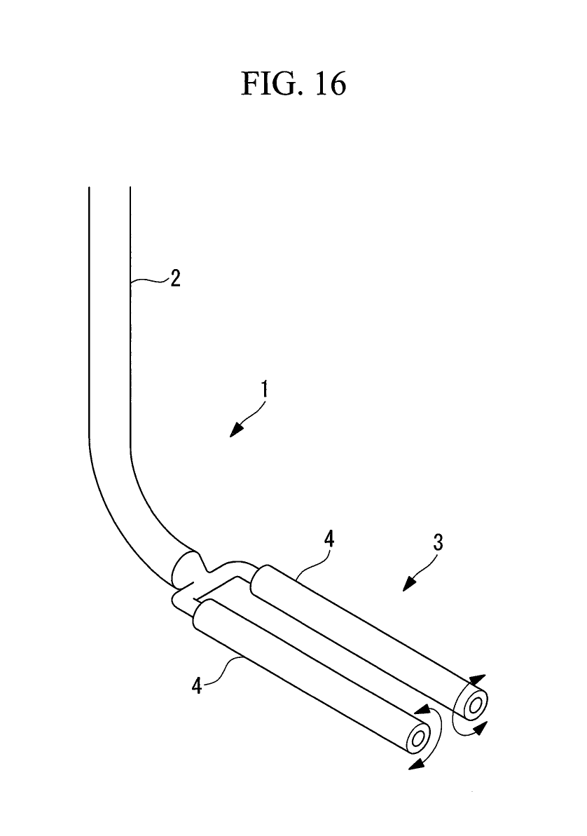

Although the contact portions 4 have been described as having a simple bar shape as an example, contact portions 4 formed of rollers rotatable about the longitudinal axes may be employed instead, as shown in FIG. 16. According to this structure, when the contact portions 4 pinching the atrial appendage A in the thickness direction are moved toward the tip or base of the atrial appendage A, this movement is made smooth by rotating the contact portions 4 formed of rollers. Rotating the rollers can reduce the friction with respect to the atrial appendage A and can decrease the load applied to the surface of the atrial appendage A.

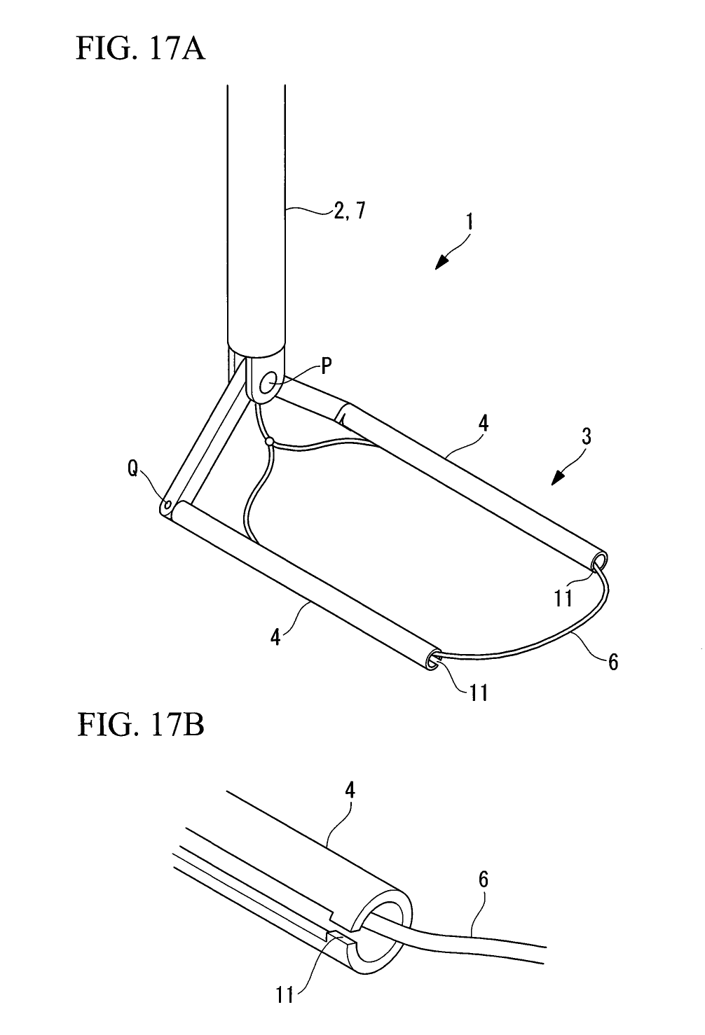

In this embodiment, the surgical tool 1 for atrial appendage ligation has been described as being a separate tool from the litigation loop 6. Alternatively, as shown in FIG. 17A, the litigation loop 6 may be integral with the surgical tool 1.

In the example shown in FIG. 17A, the shaft 2 of the surgical tool 1 for atrial appendage ligation is hollow so as to also serve as a loop shaft 7, and a retention portion 11 that retains a part of the litigation loop 6 is provided at each of the distal ends of the contact portions 4.

As shown in FIG. 17B, the retention portion 11 is formed to have a ring shape that allows the ligation loop 6 to pass through, and a slit that has a width slightly smaller than the outer diameter of the ligation loop 6 is formed therein. According to this structure, when the ligation loop 6 is not tightened, the retention portion 11 retains the ligation loop 6 inside the hollow contact portion 4, and because the spacing between the contact portions 4 is wide, the ligation loop 6 is wide open and it becomes easier to insert the atrial appendage A. When the ligation loop 6 is tightened to ligate the atrial appendage A, the ligation loop 6 passes through slits of the retention portions 11 due to tension thereof and is released from the retention by the retention portions 11.

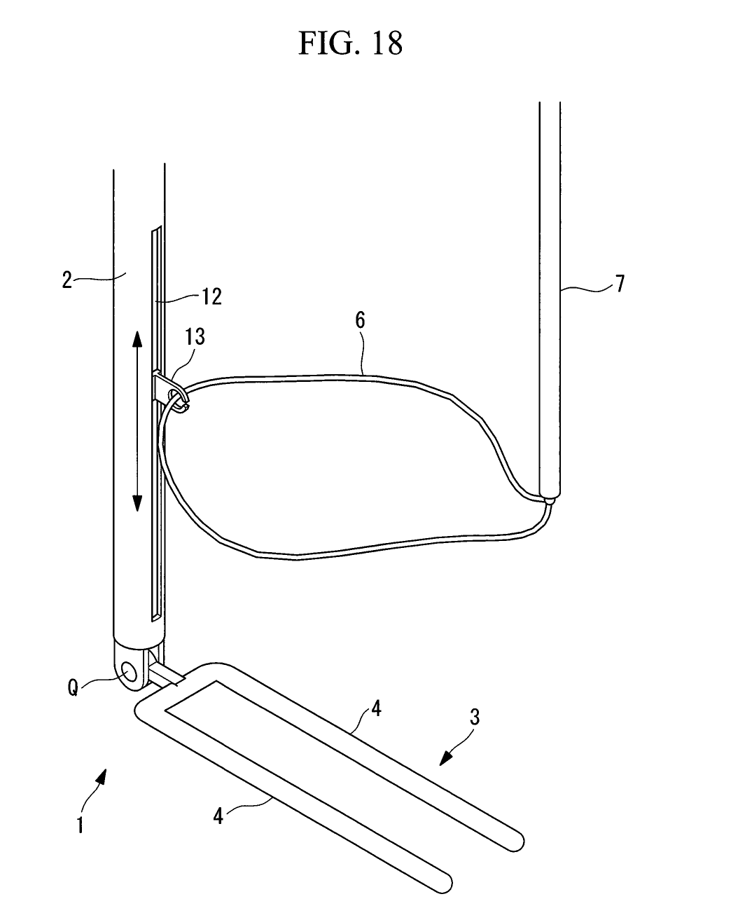

As shown in FIG. 18, a rail 12 may be disposed in the longitudinal direction of the shaft 2 of the surgical tool 1 for atrial appendage ligation so that a retention member 13 that retains a part of the ligation loop 6 can be moved along the rail 12.

In this case, when the ligation loop 6 is put, by using the loop shaft 7, around the atrial appendage A to which the shaft 2 is fixed by being pinched by the contact portions 4 of the surgical tool 1 for atrial appendage ligation, the ligation loop 6 can be moved by using the retention member 13 while maintaining a wide open state. Although the atrial appendage A changes in position due to pulsation, the shaft 2 fixed to the atrial appendage A moves together with the atrial appendage A. Thus, the ligation loop 6 can be stably put around the atrial appendage A, along the rail 12 of the shaft 2.

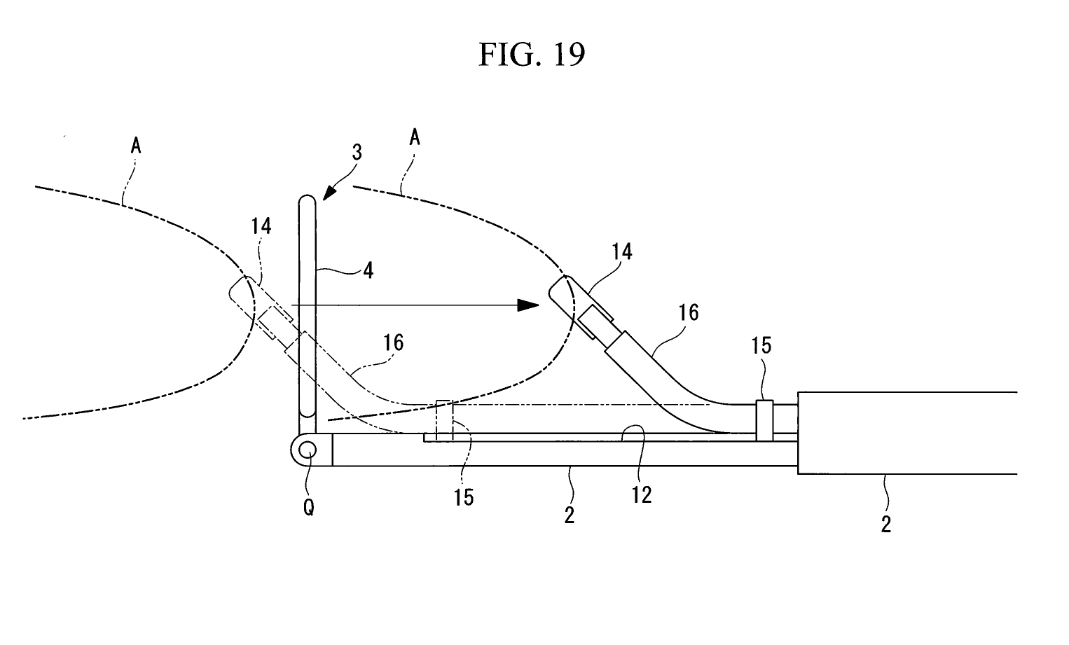

As shown in FIG. 19, the surgical tool 1 for atrial appendage ligation and retention forceps 14 may be installed so as to be relatively movable in the longitudinal direction along the rail 12 disposed in the shaft 2. Reference numeral 15 denotes a slider.

In other words, while the shaft 2 is pushed forward to press the base of the atrial appendage A with the pressing part 3 of the surgical tool 1 for atrial appendage ligation, the tip of the atrial appendage A pinched by the retention forceps 14 is pulled toward the proximal side so as to stretch the atrial appendage A and expose the base so that the ligation loop 6 can be easily placed therearound. Accordingly, the operation of the surgical tool 1 for atrial appendage ligation and the operation of the retention forceps 14 occur on the same straight line, and since they are installed to be relatively movable, ease-of-operation can be improved.

In this case, a forceps shaft 16, which supports the retention forceps 14, may have a pre-bent section so that the forceps shaft 16 is configured to deform in a direction slightly away from the shaft 2 of the surgical tool 1 for atrial appendage ligation when it is protruded forward from the sheath 5. This is because these shafts are arranged closed to each other within the sheath 5, but the tip of the atrial appendage A can be more easily retained if the shafts are slightly distant from each other.

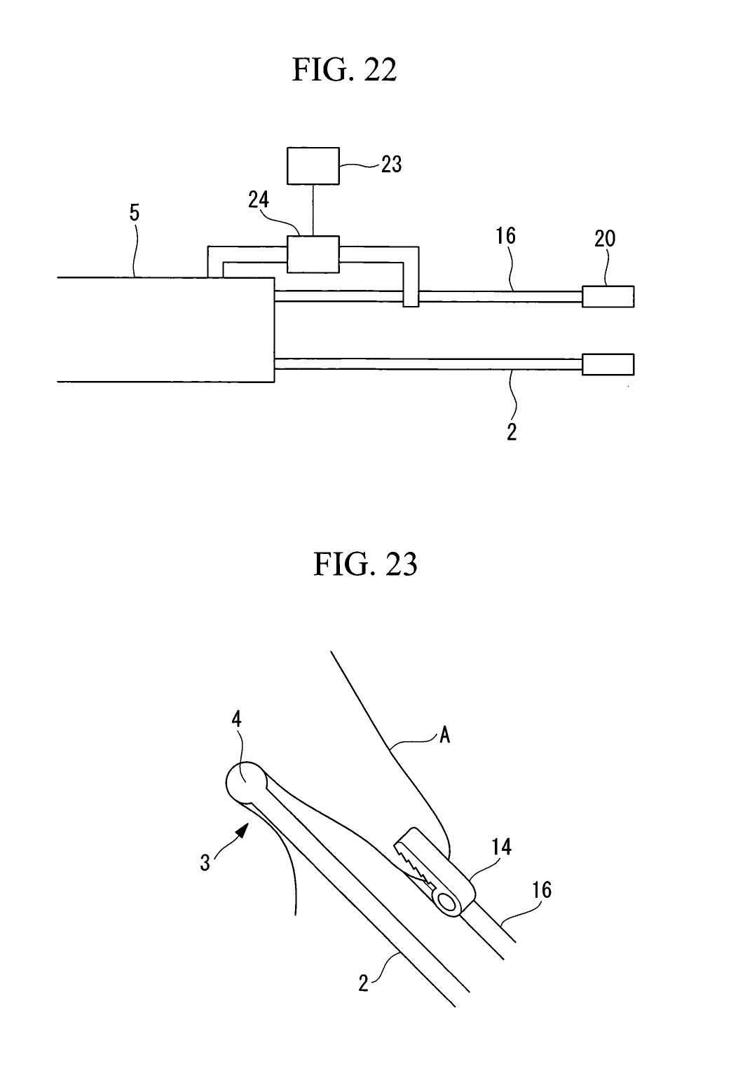

In this case, as shown in FIG. 22, an actuator 24 connected to an electrocardiographic monitor 23 may be used so that the forceps shaft 16 and the retention forceps 14 are movable in the longitudinal direction in response to a characteristic signal from the electrocardiographic monitor. For example, the timing at which the left atrium expands and which is calculated from the waveform of the electrocardiograph is used as a trigger to drive the forceps shaft 16 to relatively withdraw the pressing part 3 toward the proximal end side relative to the shaft 2. In this manner, it becomes possible not to apply excessive pressure to the left atrium by the pressing tool 3 when the left atrium is expanded.

The pressing tool 3 may be configured to immediately move toward the proximal end side as soon as a hemodynamic abnormality (ventricular fibrillation or atrial fibrillation) is detected from the waveform of the electrocardiograph. According to this configuration, it becomes possible not to apply excessive pressure to the left atrium by the pressing tool 3.

In this case, a stopper (not shown in the drawings) may be provided on the proximal end side of the surgical tool 1 and the retention forceps 14, which are connected via a slider 15. While pressing the base of the left atrial appendage with the pressing part 3 and stretching the left atrial appendage with the retention forceps 14, the positions of the shafts of the surgical tool 1 and the retention forceps 14 are locked. As a result, the force of the stretched atrial appendage trying to return to the unstretched state can be decreased, the exposed state can be maintained, and thus ligation can be facilitated.

As shown in FIG. 21, the shaft 2 of the surgical tool 1 for atrial appendage ligation and the forceps shaft 16 of the retention forceps 14 may be configured to be capable of coordinated movement in the longitudinal direction parallel to each other.

The procedure according to this configuration is described next.

First, when the left atrial appendage is retained with the retention forceps 14, the pressing tool 3 of the surgical tool 1 for atrial appendage ligation is disposed on the proximal end side of the jaws of the retention forceps 14. Next, the tip of the atrial appendage is retained by the retention forceps 14, and a proximal side operation unit 20 of the retention forceps 14 is pulled toward the proximal end side. As a result, the shaft 2, which is engaged with a gear 22 of a connecting portion 21, of the surgical tool 1 protrudes forward due to the movement of the gear 22. By this operation, pulling the retention forceps 14 toward the proximal end side and pressing the atrial appendage with the pressing part 3 can be easily carried out in one action, and the operation time can be shortened.

In this embodiment, an example of the contact portions 4 that indent the surface of the atrial appendage A with the pressing force has been described. Alternatively, the contact portions 4 may be suction pads, and the atrial appendage A may be constricted by suctioning the surface of the atrial appendage A.

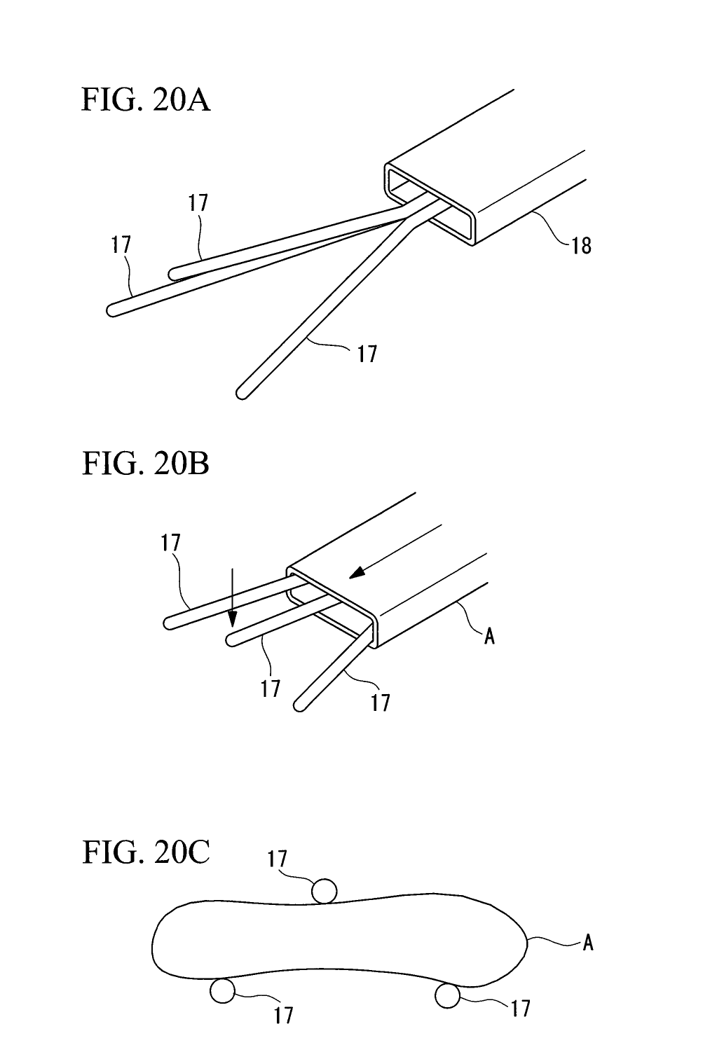

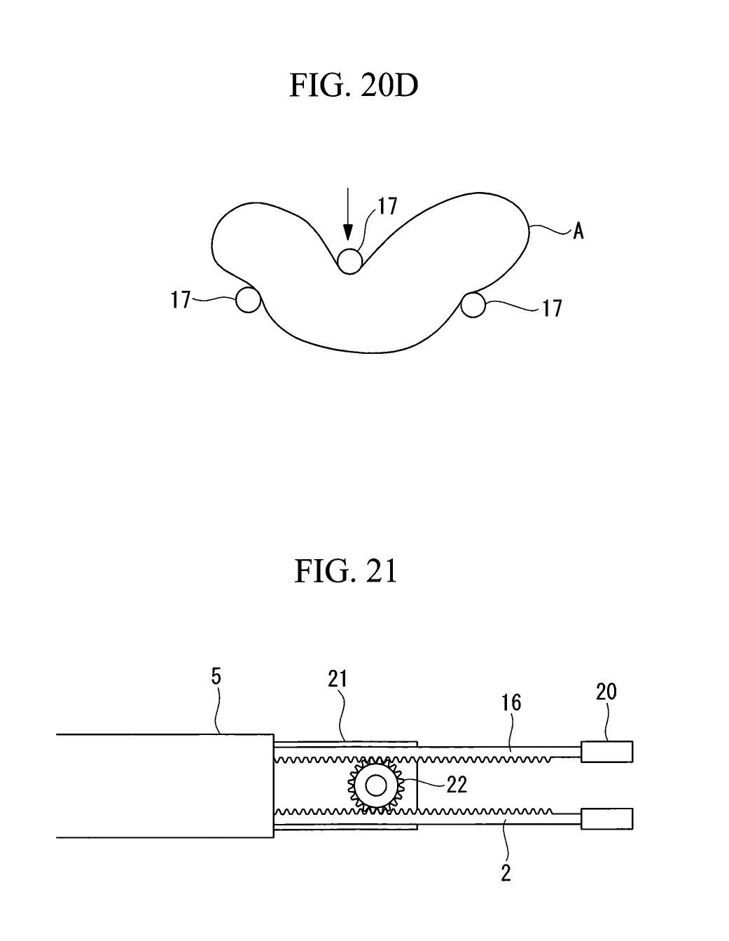

Another surgical tool that can be employed is a surgical tool 1 for atrial appendage ligation in which the spacing between three bar-shaped contact portions, which are separated from one another as shown in FIGS. 20A and 20C, is decreased by advancing a sleeve 18 as shown in FIGS. 20B and 20D, so as to fold the atrial appendage A pinched by the contact portions 17 and to thereby deform the atrial appendage A into a cross-sectional shape that facilitates ligation with the ligation loop 6.

In this embodiment, a pressing part 3, which has two contact portions 4, has been described as an example. Alternatively, as shown in FIG. 23, a pressing part having one contact portion 4, which has no sharp distal end and has a shape that does not damage the tissue on the surface of the heart, may be employed. The shaft 2 may be the same as the one shown in FIG. 23, and the pressing part 3 may be configured to have a contact portion 4 that has more than one branch and is configured to press the atrial appendage at more than one position.

### REFERENCE SIGNS LIST

A atrial appendage
1 atrial-appendage ligation surgical tool
2 shaft (biasing means)
3 pressing part
4 contact portion
5 sheath
6 ligation loop
8 distal end (magnetic material)
9 electromagnet
11 retention portion
13 retention portion (retention member)

1. An atrial-appendage ligation surgical tool, comprising:
a pressing part that is guided into a pericardium via a sheath penetrating through a pericardial membrane, and that includes a contact portion that presses a vicinity of a base of an atrial appendage; and
a shaft that is disposed in an inserted state in the sheath and that supports the pressing part at a distal end.

2. An atrial-appendage ligation surgical tool comprising:
a pressing part that is guided into a pericardium via a sheath penetrating through a pericardial membrane, and that includes two bar-shaped contact portions that can be arranged along a width direction of the atrial appendage and at a position where the atrial appendage is pinched in a thickness direction;
a shaft that is disposed in an inserted state in the sheath and that supports the pressing part at a distal end; and
a biasing means for biasing the pressing part so that the pressing part is arranged in lined manner with respect to the shaft when the pressing part is inside the sheath and the pressing part extends in a direction intersecting a longitudinal axis of the shaft when the pressing part is released from the sheath.

3. The atrial-appendage ligation surgical tool according to claim 2, wherein the contact portions are configured so that a spacing therebetween is changeable.

4. The atrial-appendage ligation surgical tool according to claim 2, wherein the contact portions are rollers that are rotatable about longitudinal axes thereof.

5. The atrial-appendage ligation surgical tool according to claim 2, wherein the shaft is made of an elastic material and has a pre-bent section to constitute the biasing means.

6. The atrial-appendage ligation surgical tool according to claim 3, wherein one of the contact portions includes a magnetic material, and the other contact portion includes a magnet.

7. The atrial-appendage ligation surgical tool according to claim 2, wherein the two contact portions have retention portions that releasably retain a ligation loop guided along the shaft.

8. The atrial-appendage ligation surgical tool according to claim 1, wherein a retention portion that releasably retains a ligation loop is provided so as to be movable along the shaft.

9. An atrial appendage ligation system comprising: retention forceps that retain an atrial appendage; and the surgical tool for atrial appendage ligation according to claim 1.

10. The atrial appendage ligation system according to claim 9, wherein the retention forceps and the surgical tool are provided so as to be relatively movable in a longitudinal direction.

11. The atrial-appendage ligation surgical tool according to claim 2, wherein a retention portion that releasably retains a ligation loop is provided so as to be movable along the shaft.
